(19) United States
(12) Patent Application Publication
(10) Pub. No.: US 2017/0000883 A1
(43) Pub. Date: Jan. 5, 2017

(54) SUPERANTIGEN POST-EXPOSURE THERAPY

(71) Applicant: THE SECRETARY OF STATE FOR DEFENCE, Wiltshire (GB)

(72) Inventors: Alun James CARTER, Salisbury Wiltshire (GB); Sarah Joanne Christine WHITFIELD, Salisbury Wiltshire (GB)

(21) Appl. No.: 15/105,160

(22) PCT Filed: Dec. 9, 2014
(86) PCT No.: PCT/GB2014/000509

§ 371 Date: Jun. 16, 2016

(30) Foreign Application Priority Data
Dec. 16, 2013 (GB) 1322209.6

(51) Int. Cl. A61K 39/395; G01N 33/50; G01N 33/68; C07K 16/28; C07K 14/705

(52) U.S. Cl. CPC A61K 39/3955; C07K 16/2827; C07K 14/70521; G01N 33/6869; G01N 33/6866; G01N 33/5014; A61K 2039/545

(57) ABSTRACT

The present invention relates to a post-exposure therapeutic against superantigen-mediated disease in a subject.

The present invention relates to a post-exposure therapeutic against superantigen (SAg)-mediated disease in a subject, in particular a post-exposure therapeutic against Staphylococcus Enterotoxin B.

The presentation of antigen to T-cells during the host adaptive immune response can occur via the interaction between the Major Histocompatibility Complex (MHC) class II, expressed on the surface of an antigen presenting cell (APC), and the T-cell receptor (TCR), expressed on the surface of a T-cell. Under normal conditions, the reversible interaction between MHC class II, TCR and presented antigen results in T-cell activation, the hallmarks of which are intracellular signalling, T-cell proliferation and induction of pro-inflammatory cytokines. The Akt pathway is the principle intracellular signalling pathway for T-cell activation, activating NFkappaB which subsequently translocates to the T-cell nucleus via the nucleoporin Nup88. Once in the nucleus, NFkappaB up-regulates many cytokines associated with inflammation. A secondary co-stimulatory signal through the APC B7 family receptors (for example B7-1 and B7-2; also termed Cluster of differentiation (CD) 80 and CD86 respectively) and T-cell CD28 receptor is also required to complete T-cell activation. A state of T-cell tolerance or unresponsiveness (T-cell anergy) can arise, for example, as a result of T-cell activation without the associated co-stimulatory signal.

In order for the immune system to regulate its activity, mechanisms for up-regulating host adaptive immune responses to potentially foreign antigens must be balanced by mechanisms for down-regulating host adaptive immune responses where, for example, foreign antigens are no longer present in the host system. One such T-cell mechanism for down-regulation of immune processes is based on the Cytotoxic T-Lymphocyte Antigen 4 (CTLA4; also known as CD152). CTLA4 is a T-cell receptor capable of binding to APC B7 receptors. However, in contrast to CD28, which upon binding to CD80 or CD86 promotes T-cell activation, CTLA4 binding to the APC B7 receptors promotes inhibition of T-cell activation, thus dampening the normal host adaptive immune response.

Superantigens (SAgs) are a family of secreted protein toxins characterised by a property of extreme potency for activation of the host adaptive immune system. SAgs are primarily considered a virulence factor of certain bacterial species, although mycoplasma, viruses and mycoplasma are also associated with SAg production. Recent studies have implicated SAgs as a principle driver for immune-dysregulation in severe sepsis patients. Since first being described in 1989, the biological toxicity exerted by SAgs is known to result from the damaging effects of rapid overstimulation of a host's own T-cells. A feature of SAgs are the low levels required for toxicity; although the mitogen potency can vary, some SAgs are capable of stimulating human T-cells in vitro at a concentration of 1 femtogram/ml (10⁻¹⁵ g/ml) (Fraser J. D. & Proft T. 2008. The bacterial superantigen and superantigen-like proteins. Immunological Reviews. 225: 226-243).

SAgs elicit their toxic activity by bridging the immune synapse between the APC and T-cell via fusing of the MHC class II and TCR. In particular, all known SAgs are thought to bind to a subset of the variable region of the TCR β-chain. As a result of MHC class II-TCR binding, the activation of the T-cell population via TCR signalling promotes a subsequent rapid release of high levels of cytokines (‘cytokine storm’). SAg interaction with T-cells may result in >20% activation of the T-cell populations, whilst the release of pro-inflammatory cytokines such as interleukin-2 (IL-2), interferon-γ (IFN-γ) and tumour necrosis factor-α (TNF-α) are commonly associated with SAg toxicity (Fraser J. D. & Proft T. 2008).

The massive immune response and subsequent immunocompromised status of intoxicated subjects, due to T cell anergy and/or depletion of immune cells, can lead to a SAg-mediated disease condition termed Toxic Shock Syndrome (TSS). TSS is associated with host systemic SAg intoxication and is characterised by symptoms including fever, headache, diarrhoea, vomiting, erythematous rash and, in more severe cases, hypotension shock, respiratory distress syndrome, intravenous coagulation, severe thrombocytopenia, renal failure and death (Fraser J. D. & Proft T. 2008). Other diseases associated with bacterial SAgs are infective endocarditis, atopic dermatitis, allergic rhinitis and autoimmune disease such as Reactive Arthritis (RA) and Graft Versus Host Disease (GVHD).

Research into SAgs has, in addition to defining the common biological mechanism for toxicity, identified a range of bacteria that deploy SAgs as part of their suit of virulence factors. For example, the bacterium Yersinia pseudotuberculosis is thought to produce, three SAg variants, termed Yersinia tuberculosis Mitogen-A, B and C respectively. However, one of the primary bacterial species associated with SAgs is Staphylococcus aureus, known to produce at least 20 serologically different SAgs comprising Staphylococcal enterotoxin A (SEA) and Staphylococcal enterotoxin B (SEB), associated with food poisoning, and TSS Toxin-1 (TSST-1), associated with TSS. Further identified Staphylococcus exotoxins (SEs) are the individual variants C-E and G-J, as well as Staphylococcus enterotoxin-like toxins (SEI) K-R and U, U2 and V with undefined enterotoxicity.

Another bacterium associated with the productions of SAgs is Streptococcus pyogenes. To date, more than 10 Streptococcal SAgs have been identified: SPE-A, -C, -G, -H, -I, -J, -K/L, -L/M, -M, SSA, SMEZ-1 and SMEZ-2. S. pyogenens SAgs are associated with a number of diseases, including: Scarlet fever, as a result of pharyngeal Streptococcus infection; acute rheumatic fever in the young; and myositis. In particular, the SMEZ Streptococcal SAgs have a particularly high T-cell mitogenic activity (<0.1 pg/ml), possibly accounting for S. pyogenes being associated with a more acute form of TSS, with higher fatality rate than that of S. aureus-associated TSS, as well as the notorious flesh-eating bacterial syndrome necrotizing fasciitis.

Current treatment for SAg-mediated disease is limited to supportive care of subjects, requiring utilising critical intensive care resources that include administration of fluids, inotropes and ventilation, and antibiotic therapy in the case of sepsis. In particular, the resultant effect of incapacitation following SAg-mediated disease creates a potentially very high logistical casualty care burden.

One therapeutic strategy explored as therapeutic intervention of SAg-mediated disease is the administration of intravenous immunoglobulin (IVIG), comprising highly polyspecific antibodies, which act to neutralise SAg toxic activity. This approach is based on antibody serum neutralizing activity being a key factor for reducing the risk of toxic SAg effects. However, IVIG treatment of SAg-mediated disease has had varied results and, due to the high mitogenic potency of SAgs, requires high levels of administered IVIG. Furthermore, this approach may only benefit those treated in the early stages of SAg-mediated disease. This issue highlights a problem with SAg-mediated disease in that the therapeutic window for medical intervention is thought to be short due to the speed of the onset of the inflammatory response.

Employing peptide antagonists with the aim of preventing binding of SAg with MHC class II-TCR complexes has been studied. However, use of SEB fragments failed to prevent the effects of SEB in vitro or in vivo, using human T-cells and transgenic mice respectively (Rajagopalan et al. 2004. In Vitro and In Vivo Evaluation of Staphylococcal Superantigen Peptide Antagonists. Infection and Immunity. 72:6733-6737).

Efforts have assessed the use of anti-inflammatories as a therapeutic for SAg-mediated disease, in light of the correlation between toxicity and host levels of released pro-inflammatory cytokines. Two recent studies demonstrated that dexamethasone could improve survival when administered post SEB exposure. However in the first study dexamethasone was only effective at attenuating the toxic effects of SEB when given 4.25 hours post exposure and required further therapeutic intraperitoneal administration at 20 hours and 24 hours (80% survival in the treatment group) (Krakauer T. & Buckley M. 2006. Dexamethasone Attenuates Staphylococcal Enterotoxin B-Induced Hypothermic Response and Protects Mice from Superantigen-Induced Toxic Shock. Antimicrobial Agents and Chemotherapy. 50:391-395). The second study required an intranasal administration of dexamethasone 3 hours post SEB exposure and also required intraperitoneal dose every 24 hours for 4 additional days (70% survival in the therapeutic group) (Krakauer T. et al. 2009. Critical timing, location and duration of glucocorticoid administration rescue mice from superantigen-induced shock and attenuate lung injury. International Immunopharmacology. 9:1168-1174). Greater success was observed when rapamycin was administered to mice, via the intranasal route, 5 hours post exposure to SEB (Krakauer et al. 2010. Rapamycin Protects Mice from Staphylococcal Enterotoxin B-induced Toxic Shock and Blocks Cytokine Release In Vitro and In Vivo. Antimicrobial Agents and Chemotherapy. 54:1125-1131). However, additional intraperitoneal administration of rapamycin every 24 hours for 4 further days was required to demonstrate drug efficacy. Studies have alternatively utilised humanised MHC class II receptor transgenic murine models to establish the toxicity of SEB and the potential efficacy of therapeutic compounds. Although an SEB antitoxin used in conjunction with statins (lovastatin) demonstrated 70% survival in one transgenic mouse model, their utility as a SEB therapeutic remains to be elucidated as the therapies were administered at the same time as SEB exposure (Tilahun et al. 2011. Chimeric Anti-Staphylococcal Enterotoxin B Antibodies and Lovastatin Act Synergistically to Provide In Vivo Protection against Lethal Doses of SEB. PLOS ONE. 6:1-8).

Such investigations thus demonstrate inherent problems with compounds for SAg therapeutics, and in particular SEB therapeutics. For example, compounds studied to date require multiple treatment administrations, intranasal dosing and/or treatment within a short therapeutic window. This poses several logistical medical burdens, for example administration of multiple doses of therapy, the administration of the drugs via the inhalational route and a short therapeutic window of opportunity.

Thus, there is a requirement for an effective post-exposure therapeutic for treatment for SAg-mediated disease. The present invention thus aims to address this problem.

Accordingly, in a first aspect of the present invention there is provided a pharmaceutical composition for use, in the post-exposure treatment of SAg-mediated disease in a subject, wherein the pharmaceutical composition comprises an active ingredient which is capable of binding an antigen presenting cell B7 receptor.

As used herein, post-exposure relates to administration after the subject has been exposed to a SAg.

The pharmaceutical composition may be administered in multiple post-exposure doses i.e. at least one post-exposure dose. Preferably however, the pharmaceutical composition is administered as a single dose, wherein the single dose is administered post-exposure.

The Applicant has surprisingly found that drugs/therapeutics that target APC B7 receptors are efficacious for post-exposure treatment of SAg-mediated disease in a subject, and,in particular that protection can be provided by a single post-exposure dose. This is advantageous as pre-exposure use of such a pharmaceutical composition is impractical, given that a subject would be unlikely to anticipate the requirement for treatment of SAg-mediated disease and that the therapeutic window for medical intervention is thought to be short due to the speed of the onset of the inflammatory response.

The term SAg intoxication in relation to the present invention includes, but not exclusively, exposure to the superantigen in its pure form or septic shock resulting from pathogens expressing SAg.

The term subject in relation to the present invention includes any animal. In particular, an animal in need or thought to be in need of being administered a pharmaceutical composition for use in the post-exposure treatment of SAg-mediated disease.

The term B7 receptors in relation to the present invention includes receptors comprising the B7 receptor family, for example B7-1 (also known as CD80) and B7-2 (CD86), B7-H1 (Programmed Cell Death Ligand-1 (PD-L1) or CD274), B7-DC (PD-L2), B7-H2 (L-ICOS), B7-H3 and B7-H4.

Pharmaceutical composition active ingredients which are capable of binding to APC B7 receptors include CTLA4, CTLA4Ig, and derivatives thereof, and programmed death-1 and alternative B7 receptor recognition elements.

As used herein, CTLA4Ig is a fusion protein, also known as abatacept, developed by Bristol-Myers-Squibb, containing the extracellular domain of CTLA4 combined with the Fc region of immunoglobulin IgG1. Not wishing to be bound by theory, due to the presence of the CTLA4 region, CTLA4Ig is capable of binding to APC B7 receptors, and in particular B7-1 (CD80), thus preventing binding with T-cell-associated CD28 and subsequently inhibiting full T-cell activation via co-stimulatory signalling.

For the purpose of this invention, CTLA4Ig can be used interchangeably with the term abatacept.

The medicament known by the marketed trade name Orencia® comprises the active substance abatacept. Orencia® is suitable for treatment of arthritis, including rheumatoid arthritis and polyarticular juvenile idiopathic arthritis (European Public Assessment Report EMA/526106/2012). Forms of Orencia® suitable for treatment include a powder that is made up into a solution for intravenous infusion and as a solution for subcutaneous injection.

Derivates of CTLA4Ig include, but not exclusively, belatacept, a soluble fusion protein is based on CTLA4Ig but which contains two substituted amino acids in the CTLA4 ligand-binding region.

The term programmed death-1 includes, but not exclusively, the protein product of the PDCD1 gene, which is capable of binding to B7 family receptors, in particular PD-L1 and PD-L2. Similar to CTLA4Ig, PD-1 binding to B7 family receptors can negatively regulate processes associated with T-cell activation.

The term alternative B7 receptor recognition elements includes antibodies or synthetic therapeutics such as aptamers that are capable of binding to B7 family receptors, the result of which includes preventing binding of B7 family receptors with T-cell-associated CD28.

The Applicant has in particular found that pharmaceutical composition active ingredients relating to CTLA4Ig are effective in post-exposure treatment of SAg-mediated disease. Consequently, in one embodiment of the first aspect of the present invention the active ingredient is selected from CTLA4Ig or derivatives thereof.

In a particular embodiment of the first aspect of the present invention, the active ingredient is CTLA4Ig.

It is acknowledged that previous work by Saha et al. (Saha B. et al. 1996. Toxic Shock. Syndrome Toxin-1-Induced Death is Prevented by CTLA4Ig. The Journal of Immunology. 157:3869-3875) investigated use of CTLA4Ig for reduced TSST-1-induced TSS in vivo, showing 75% improved survival in a 24 hr lethal murine model of TSST as compared to controls. However, the murine model of Saha et al. utilised a combination of a pre- and post-exposure dosages of CTLA4Ig. Saha et al. is silent as to the respective roles of the pre- and post-exposure dosages, or whether both contributed to protection or not. Saha et al. does not discuss the relevance of the post-exposure dose of CTLA4Ig. However, Saha et al. clearly suggests that for CTLA4Ig to work effectively against TSST-1 exposure, both a pre-exposure CTLA4Ig dose and a post-exposure CTAL4Ig dose would be required for any beneficial effect.

US 2008/0038273 discloses use of CTLA4Ig as a control for in vitro studies investigating TSST-mediated T-cell proliferation and does not disclose post-exposure in vivo treatment of SAg-mediated disease.

Surprisingly, the Applicants have demonstrated that pre-treatment of subjects with CTLA4Ig is not necessary for efficacy against SAg-mediated disease. Furthermore, the Applicants have shown that a single administration of CTLA4Ig up to 8 hr post-exposure was effective against SAg-mediated disease. This is advantageous as such practical administration extends the window of opportunity for treatment of SAg-mediated disease. To the Applicant's knowledge; this extended window of opportunity is better than any other SAg medical countermeasure previously reported.

The Applicant has in particular shown that CTLA4Ig is efficacious for use in the post-exposure treatment of SEB-mediated disease.

Thus, in a further embodiment of the first aspect of the present invention, the SAg is SEB.

This is particularly surprising, since Saha et al. demonstrate that CTLA4Ig-treated mice are vulnerable to challenge with SEB, concluding that murine resistance to TSST-1 as a result of CTLA4Ig administration was specific to that particular SAg.

Nestle et al (Nestle et al. 1994. Costimulation of Superantigen-Activated T-Lymphocytes by Autologous Dendritic Cells is Dependent on B7. Cellular Immunology. 156:220-229) discloses the ability of CTLA4Ig to decrease levels of SEB-mediated T-cell proliferation. However, this document is a mechanistic study focusing on in vitro experimentation, and does not disclose post-exposure in vivo treatment of SEB-mediated disease.

In a further embodiment of the first aspect of the present invention, the SAg-mediated disease is sepsis, toxic shock syndrome, infective endocarditis or necrotizing fasciitis.

In a further embodiment of the first aspect of the present invention, there is provided a pharmaceutical composition for use in the post-exposure treatment of SAg-mediated disease in a subject, wherein the pharmaceutical composition is administered via the intravenous route. Alternatively, the pharmaceutical composition is administered via the subcutaneous route.

These embodiments of the present invention are advantageous as they provide an easily-administered route of delivery for a pharmaceutical composition for use in the post-exposure treatment of SAg-mediated disease in a subject. Furthermore, it is particularly surprising, in light of the prior art, that the present invention is efficacious using a single intravenous administration rather than multiple doses across a number of days.

In a further embodiment of the first aspect of the present invention, there is provided a pharmaceutical composition for use in the post-exposure treatment of SAg-mediated disease in a subject, administered at least 3 hours post-exposure to SAg. Alternatively, there is provided a pharmaceutical composition administered at least 8 hours post-exposure to SAg. Furthermore, the pharmaceutical composition is administered at least 144 hours post-exposure to SAg.

The Applicant has shown that the pharmaceutical composition can be administered 3 hours post-exposure, and also 8 hours post-exposure, and successfully treat SAg-mediated disease. Indeed the Applicant has reasoned that the pharmaceutical composition could be delivered up to 144 hours post-exposure, based on the concentration of key biomarkers. To the Applicant's knowledge, CTLA4Ig is the first post-exposure drug to be effective against SAg-mediated disease at 8 hrs post SAg exposure, with complete mitigation of the incapacitating effects of SEB, thus extending the therapeutic window for medical intervention.

In the second aspect of the present invention, there is provided an agent capable of binding an antigen presenting cell B7 receptor for use in the post-exposure treatment of SAg-mediated disease.

In a further embodiment of the second aspect of the present invention, the agent is administered as a single dose.

In a further embodiment, the agent is CTLA4Ig, and derivatives thereof.

In the third aspect of the present invention, there is provided a method for monitoring subject responsiveness to post-exposure treatment of SAg-mediated disease with a pharmaceutical composition according to the first aspect of the present invention, the method comprising the steps of: measuring the concentration of at least one of IFN-γ and IL-6 in a first and a second biological sample, wherein the first biological sample is from a subject pre-treatment, and the second biological sample is post-treatment; comparing the pre- and post-treatment concentration of each respective biomarker; and wherein a respective biomarker concentration lower in the post-treatment biological sample, relative to the pre-treatment biological sample, is a positive indicator of subject responsiveness to post-exposure treatment.

The term biological sample in relation to the present invention includes, but not exclusively, CSF, blood or samples derived thereof (for example whole blood, plasma, serum, cell-free serum, cell-free plasma), tissues, cells, saliva, transpired secretion, urine, faeces, stomach fluid, digestive fluid, nasal fluid, cytosolic fluid or other biological tissue or fluid sample recognised in the art. Alternatively, the term biological sample includes those samples indicative of the systemic (endocrine) immune response in subjects pre- and post-treatment.

The term post-treatment in relation to the present invention includes, but not exclusively, a duration of at least three days post-treatment.

The Applicant has shown that IFN-γ and IL-6, either individually or in combination, are particularly effective at indicating subject responsiveness to post-exposure treatment for SAg intoxication, in particular SEB intoxication.

The present invention will now be described with reference to the following non-limiting examples and figures in which:

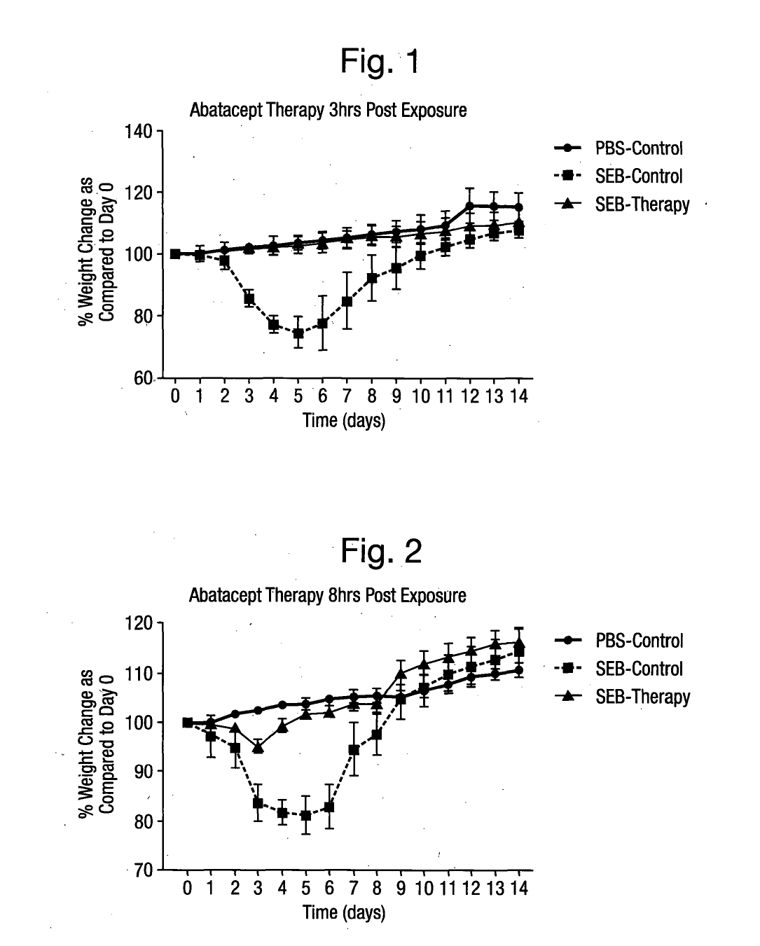

FIG. 1 is a graph showing in vivo efficacy of CTLA4Ig, administered 3 hr post SEB exposure, to mitigate SEB-induced weight loss in a sub-lethal murine model. Weight change is represented as mean percent change of body weight as compared to mouse weights on day 1 prior to SEB exposure. Graph represents mean with error bars for 95% Cl.

FIG. 2 is a graph showing in vivo efficacy of CTLA4Ig, administered 8 hr post SEB exposure, to mitigate SEB-induced weight loss in a sub-lethal murine model. Data represents two replicate in vivo studies. Weight change is represented as mean percent change of body weight as compared to mouse weights on day 1 prior to SEB exposure. Graph represents mean with error bars for 95% Cl.

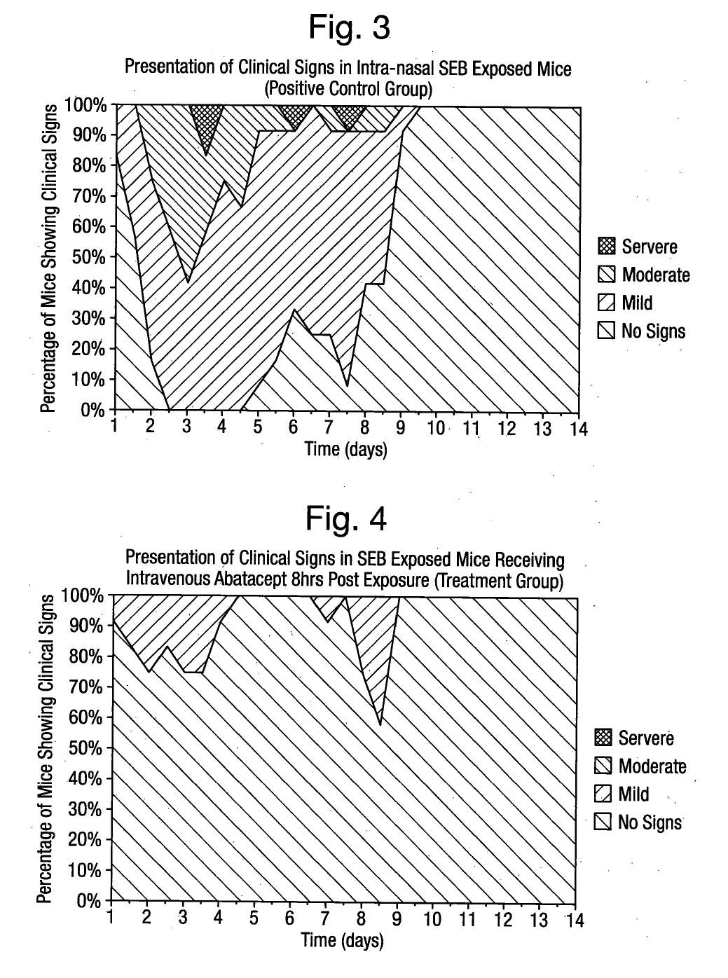

FIG. 3 is a graph showing SEB-induced clinical signs of the SEB positive control group. Graph represents percentage of mice presenting severe, moderate, mild or no clinical signs plotted for each time point.

FIG. 4 is a graph showing SEB-induced clinical signs of the SEB treated with CTLA4Ig therapy group. Graph represents percentage of mice presenting severe, moderate, mild or no clinical signs plotted for each time point.

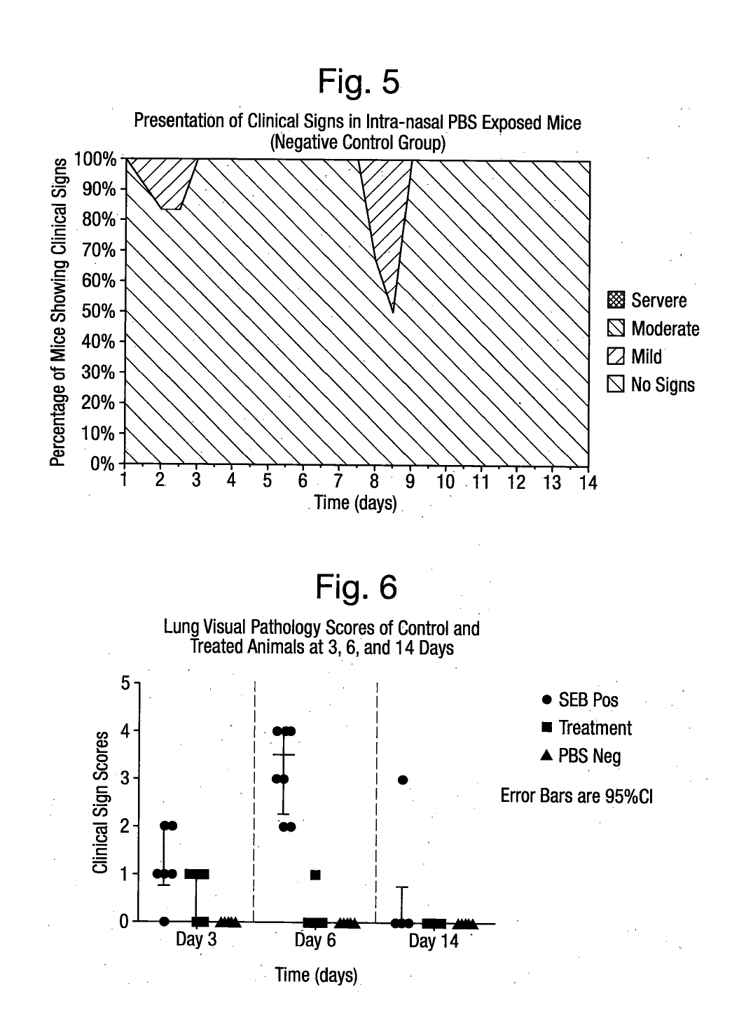

FIG. 5 is a graph showing SEB-induced clinical signs of the PBS negative control group. Graph represents percentage of mice presenting severe, moderate, mild or no clinical signs plotted for each time point.

FIG. 6 is a graph showing lung pathology scores from the SEB positive control group, SEB treated with CTLA4Ig therapy group and PBS negative control group. Scores from a minimum of 6 animals from the SEB positive control, PBS negative control and SEB treated with CTLA4Ig therapy group were measured at 3, 6 and 14 days. Graphs represent median with interquartile range.

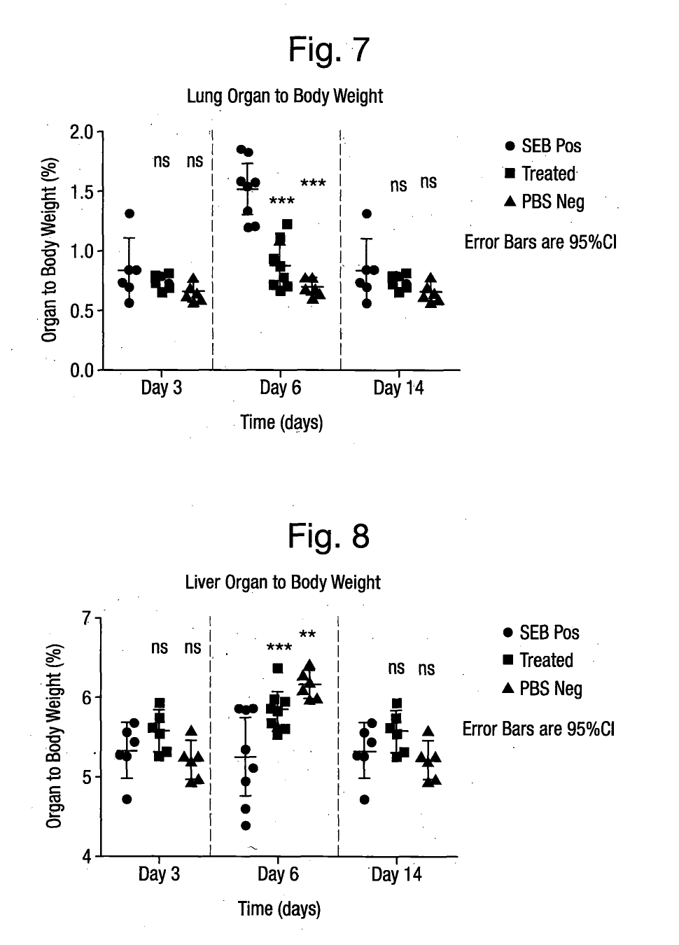

FIG. 7 is a graph showing organ to body weigh percent weight of lung in the SEB positive control group, SEB treated with CTLA4Ig therapy group and PBS negative control group. Weights were determined at 3, 6 and 14 days post SEB exposure. Graphs depict mean organ values with 95% Cl.

FIG. 8 is a graph showing organ to body weigh percent weight of liver in the SEB positive control group, SEB treated with CTLA4Ig therapy group and PBS negative control group. Weights were determined at 3, 6 and 14 days post SEB exposure. Graphs depict mean organ values with 95% Cl.

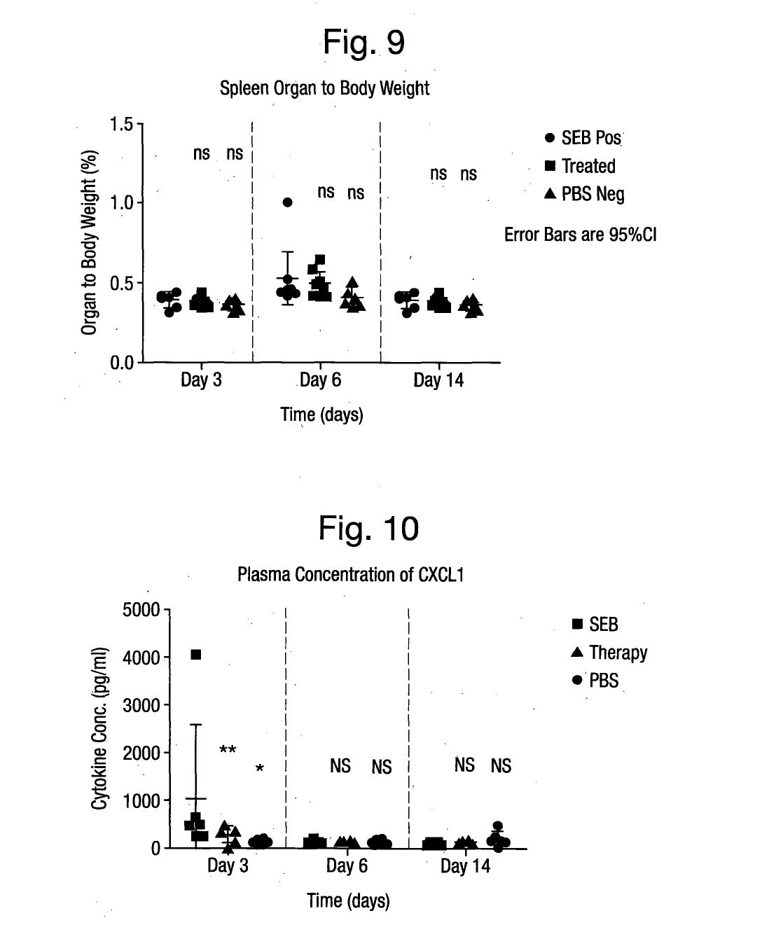

FIG. 9 is a graph showing organ to body weigh percent weight of spleen in the SEB positive control group, SEB treated with CTLA4Ig therapy group and PBS negative control group. Weights were determined at 3, 6 and 14 days post SEB exposure. Graphs depict mean organ values with 95% Cl.

FIG. 10 is a graph showing plasma concentrations of CXCL1 in the SEB positive control group, SEB treated with CTLA4Ig therapy group and PBS negative control group. Graphs depict mean cytokine values with 95% Cl.

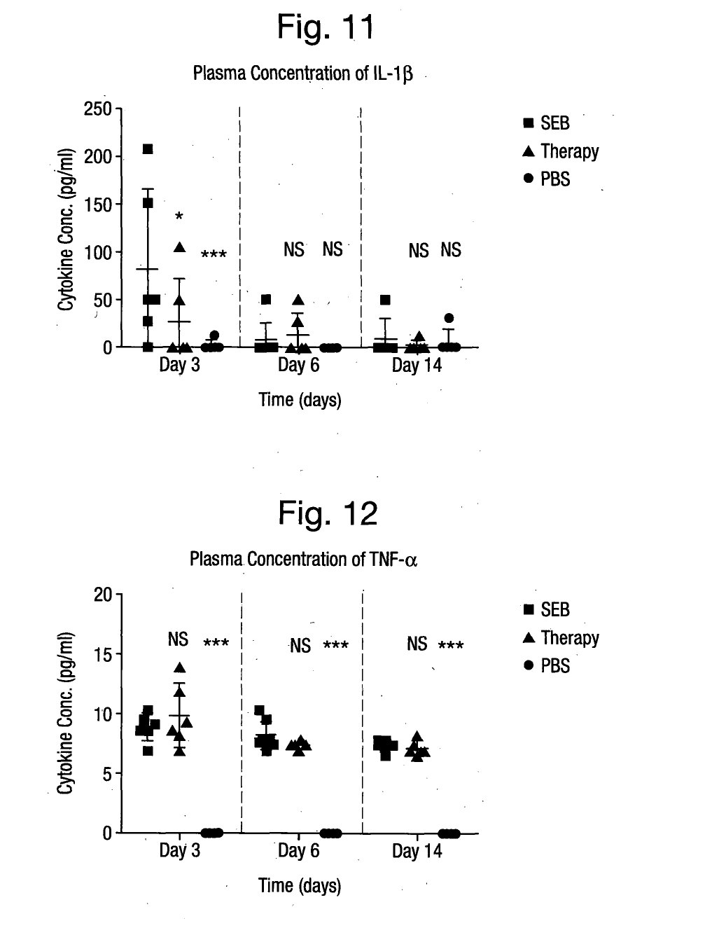

FIG. 11 is a graph showing plasma concentrations of IL-1 β in the SEB positive control group, SEB treated with CTLA4Ig therapy group and PBS negative control group. Graphs depict mean cytokine values with 95% Cl.

FIG. 12 is a graph showing plasma concentrations of TNF-α in the SEB positive control group, SEB treated with CTLA4Ig therapy group and PBS negative control group. Graphs depict mean cytokine values with 95% Cl.

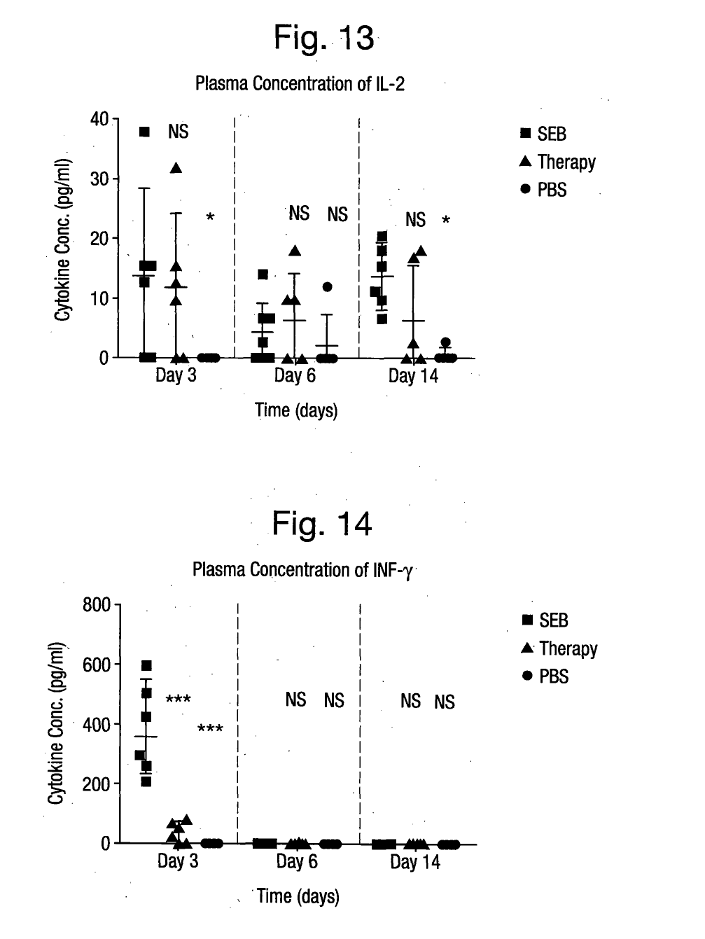

FIG. 13 is a graph showing plasma concentrations of IL-2 in the SEB positive control group, SEB treated with CTLA4Ig therapy group and PBS negative control group. Graphs depict mean cytokine values with 95% Cl.

FIG. 14 is a graph showing plasma concentrations of IFN-γ in the SEB positive control group, SEB treated with CTLA4Ig therapy group and PBS negative control group. Graphs depict mean cytokine values with 95% Cl.

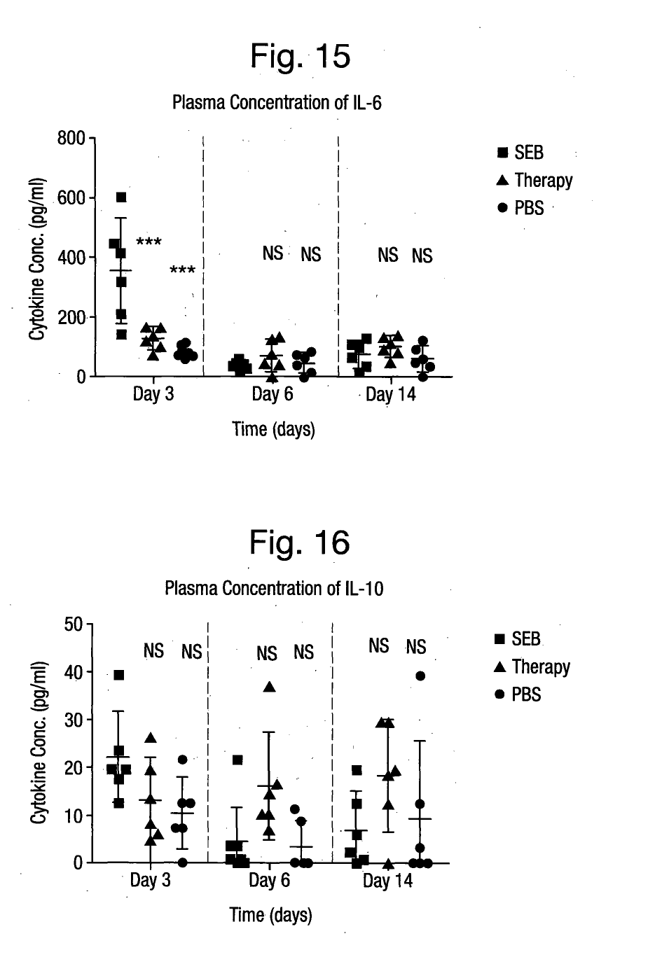

FIG. 15 is a graph showing plasma concentrations of IL-6 in the SEB positive control group, SEB treated with CTLA4Ig therapy group and PBS negative control group. Graphs depict mean cytokine values with 95% Cl.

FIG. 16 is a graph showing plasma concentrations of IL-10 in the SEB positive control group, SEB treated with CTLA4Ig therapy group and PBS negative control group. Graphs depict mean cytokine values with 95% Cl.

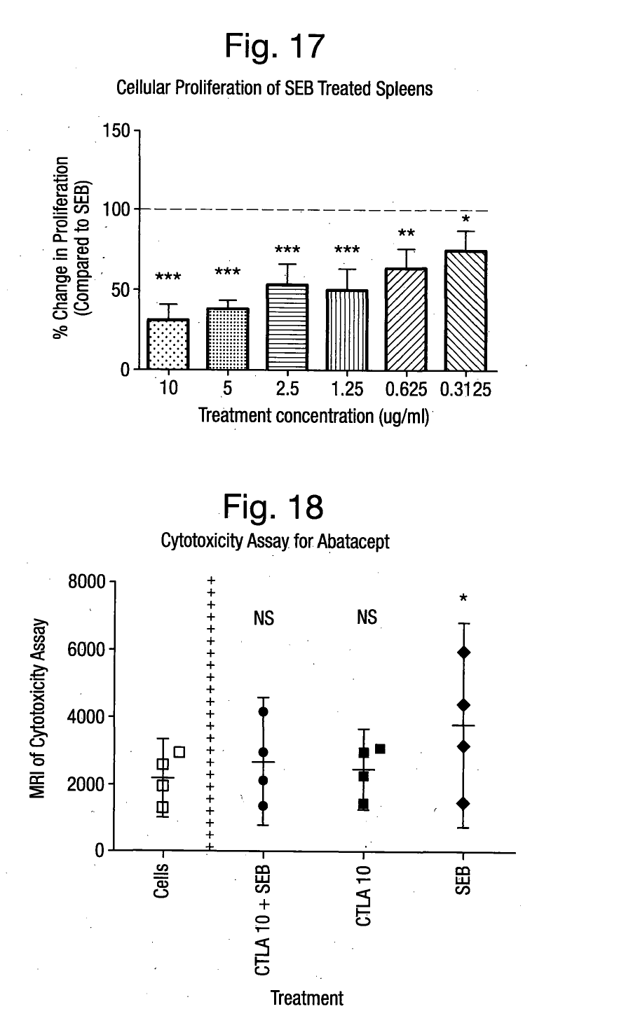

FIG. 17 is a graph showing the effect of CTLA4Ig on SEB-induced murine splenocyte proliferation. Means and 95% Cl are represented for each treatment.

FIG. 18 is a graph showing the effect of CTLA4Ig on SEB-induced murine splenocyte cytotoxicity. Cytotoxicity is presented as mean fluorescence intensity (MFI) of dead cells using a MultiTox-Fluor Multiplex cytotoxicity assay. Means and 95% Cl are represented for each treatment.

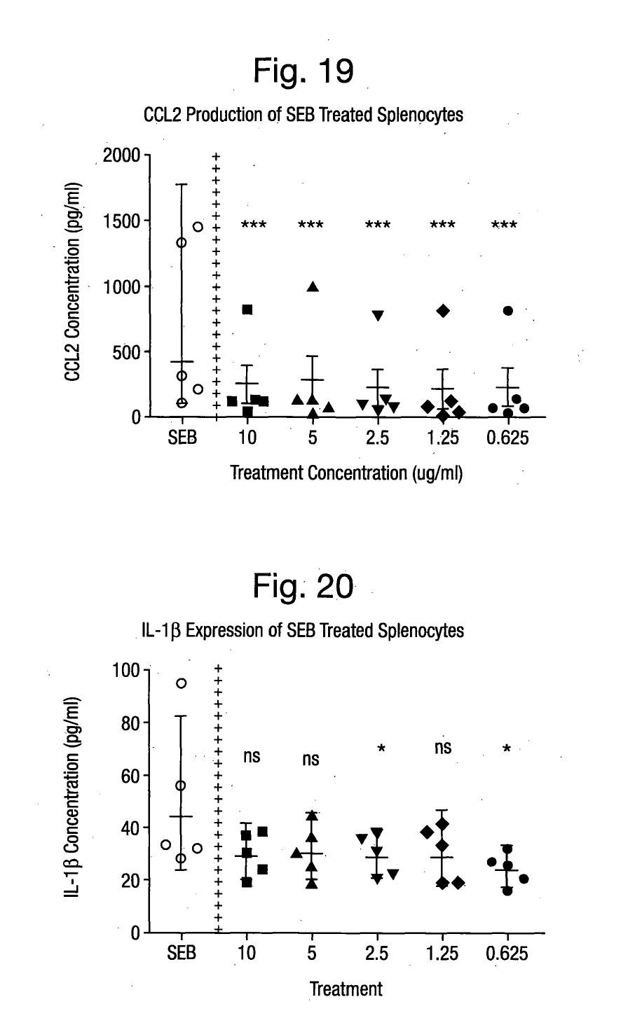

FIG. 19 is a graph showing the effect of CTLA4Ig on CCL2 expression by SEB-treated splenocytes. Means and 95% Cl are represented for each treatment.

FIG. 20 is a graph showing the effect of CTLA4Ig on IL-1β expression by SEB-treated splenocytes. Means and 95% Cl are represented for each treatment.

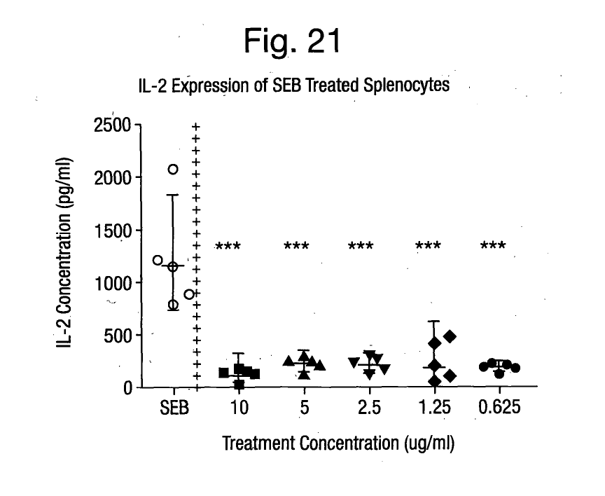

FIG. 21 is a graph showing the effect of CTLA4Ig on IL-2 expression by SEB-treated splenocytes. Means and 95% Cl are represented for each treatment.

### EXAMPLE 1

Animal Conditions

Age-matched male Balb/C mice purchased from a designated supplier (42-49 days old; Charles River Laboratories Ltd, Margate, Kent, UK) were used for all animal studies. The mice were housed in a Home Office designated establishment in rooms maintained at 21° C. +/−2° C. on a 12/12-hour dawn to dusk cycle. Humidity was maintained at 55% +/−10% with airflow of 15-18 changes/hour. Mice were kept in polycarbonate shoebox-type cages with steel cage tops and corncob bedding (International Product Supplies, Wellingborough, UK). Mice were fed a standard pelleted Teklad TRM 19% protein irradiated diet (Harlan Teklad, Bicester, UK) and given fresh water daily, ad libitum. All animals were housed according to the 1986 Scientific Procedures Act, under is appropriate ethically approved licenses from the UK Home Office.

Toxin & Therapeutics

SEB toxin was obtained from the Health Protection Agency (Porton Down, Wiltshire, UK). SEB toxin was prepared for use in all in vitro and in vivo studies at the correct concentration using 0.9% phosphate-buffered saline. CTLA4Ig (abatacept; Orencia®) (250 mg) was obtained from Bristol Myers Squibb.

In Vivo SEB Sub-Lethal (Incapacitation) Model

In vivo studies were carried out using CTLA4Ig to provide confirmation of the inhibition of SEB-induced toxicity in mice. Balb/C mice were randomly assigned to treatment or control groups and weighed on day 0 of the experiment. Intranasal administration of the SEB toxin was performed under light anaesthesia with recovery induced using Halothane (5% in the presence of 4 Lmin⁻¹ oxygen). SEB toxin used for intranasal administration was prepared as a 0.25 μg/g dose in Dubbecco's Phosphate Buffered Saline (PBS) and given as a total dose of 50 μl split between the two nares. All studies were carried out according to the Home Office Animal Licence which clearly defined humane endpoints including the loss of 30% weight loss or more and reduced mobility in the presence of severe clinical signs.

In a first study to assess efficacy of CTLA4Ig to mitigate SEB-induced weight loss, 3 groups of 6 mice were dosed as follows: SEB via the intranasal route and PBS via the intravenous route (SEB positive control groups); PBS via the intranasal and intravenous route (PBS negative control group); or SEB via the intranasal route and CTLA4Ig intravenously (10 mgkg⁻¹) (SEB treated with CTLA4Ig therapy group). Intravenous PBS or CTLA4Ig was administered 3 hr post SEB intranasal challenge. Mice were weighed daily for 14 days following SEB exposure. Statistical difference between the positive control group and CTLA4Ig therapy group was determined by a mixed linear model.

In a second study to assess efficacy of CTLA4Ig to mitigate SEB-induced weight loss, 3 groups of 1.8 mice were dosed as follows: SEB via the intranasal route and PBS via the intravenous route (SEB positive control groups).; PBS via the intranasal and intravenous route (PBS negative control group); or SEB via the intranasal route and CTLA4Ig intravenously (10 mgkg⁻¹) (SEB treated with CTLA4Ig therapy group). Intravenous PBS or CTLA4Ig was administered 8 hr post SEB intranasal challenge. Mice were weighed daily for 14 days following SEB exposure. Data represents two replicate in vivo studies. Statistical difference between the positive control group and CTLA4Ig therapy group was determined by a mixed linear model.

During both studies, the efficacy of CTLA4Ig to mitigate SEB-induced clinical signs was investigated. Data represents two replicate in vivo studies. Mice were scored twice daily for 14 days following SEB exposure, using the following scoring system for signs of SEB intoxication: 0=normal mouse; 1=slight piloerection; 2=medium piloerection; and 3=severe piloerection. The degree of abdominal pinching was also scored as mild, medium or severe. Clinical signs of intoxication were monitored according to the scoring criteria set out in Table 1. Statistical difference between the SEB positive control group and CTLA4Ig therapy group was determined by chi-squared analysis of the total number of observations for each clinical signs category.

TABLE 1
Scoring of clinical signs and mobility in the mouse.

| | | Severity |
|---|---|---|
| | | assessment |
| | | of signs |
| Score | Visible signs and mobility | of intoxication |
| 0 | None + normal mobility | Nil |
| 1 | Mild piloerection + normal mobility | Mild |
| 2 | Medium piloerection + normal | Moderate |
| | mobility | |
| 3 | Severe piloerection + normal | Severe |
| | mobility | |
| 4 | Severe piloerection + unwilling to | Severe |
| | move/reduced mobility | |
| 5 (humane end | Severe piloerection + unable to | Severe |
| point) | move | |

Summary of results: FIG. 1-5 show that CTLA4Ig treatment at 3 hr and 8 hr post SEB exposure prevents SEB-induced weight loss and clinical signs. SEB positive control group had a significant drop in weight loss (for example>15% around day 5). However, in the SEB treated with CTLA4Ig therapy group and the PSB negative control group no weigh loss was observed and indeed similar levels of weight gain demonstrated. This indicates that the CTLA4Ig completely mitigates the substantial weight loss and clinical signs induced by SEB exposure.

Pathological Assessment of Tissues

Animals were culled using cervical dislocation at either 3, 6 or 14 days following SEB intoxication. The necropsy of lung, kidney and spleen were recorded as a crude indicator for presence of atrophy, hyperplasia or tissue oedema and weighed. Lungs were examined for signs of gross pathology and the lung wet weight data recorded. The gross lung pathology severity was assessed and scored also using an increasing scale of severity from 0 (no pathology) to 4 (severe pathology) using the pre-determined scoring system set out in Table 2. Organ to body weight results, expressed as percentages of animal total body weigh at the time of culling, were assessed using a Two-way ANOVA with a Bonfferoni post test to compare the PBS negative control group and the SEB treated with CTLA4Ig therapy group to the SEB positive control group.

TABLE 2
Scoring of gross lung pathology.

| | |
|---|---|
| Score | Description |

| | | |
|---|---|---|
| 0 | Normal | Generally pale pink colouration |
| | | No hyperinflation |
| | | If present only few (<5) white |
| | | foci |
| 1 | Mild | Pale patches of congestion |
| | | Few minor focal haemorrhages |
| | | Possible hyperinflation |
| 2 | Moderate | Overall general congestion |
| | | Some hyperinflation |
| | | Froth exuding from cut trachea |
| 3 | Severe | Haemorrhagic congestion on |
| | | few lobes |
| | | Hyper inflated |
| | | Some consolidation may be |
| | | evident |
| 4 | Very severe | Haemorrhagic congestion |
| | | evident in all lung lobes with |
| | | extensive consolidation evident |

Summary of results: FIG. 6 shows that SEB exposure increased lung pathology scores at day 3 and 6. At day 6, all animals in the SEB positive control group had visible inflammatory-induced pathology of the lungs. Animals that received CTLA4Ig had reduced lung pathology scores throughout the study as compared to the SEB positive control group. Only one animal showed mild pathology of the lungs at day 6 in the SEB treated with CTLA4Ig therapy group. In addition to reduced lung pathology scores, lung to body weigh percentage was significantly lower in the SEB treated with CTLA4Ig therapy group as compared to the SEB positive control group at day 6 (FIG. 7).

Importantly, the lung weights of the SEB treated with CTLA4Ig therapy group were similar to the PBS negative control group. The results suggest that lung odema is mitigated in the therapy group when exposed to SEB as compared to animals receiving SEB and not the therapy. Visibly pathology scores and lung weights suggest that CTLA4Ig prevents SAg-induced immunopathology of the lungs.

Significant differences were observed in liver weight at day 6 between the SEB positive control group verses the SEB treated with CTLA4Ig therapy group and the PBS negative control group (FIG. 8), which may have implications in the systemic response to SAg. There were no significant changes in the weight of the spleens across all groups (FIG. 9).

CBA for Plasma and Tissues

At days 3, 6 and 14 days post SEB exposure, whole blood from 6 mice per study group was sampled into EDTA tubes. Samples were rolled for 5 min at room temperature prior to being centrifuged at 1000 ×g for 15 min at 4° C. Plasma supernatant was subsequently removed and stored prior to luminex® analysis. Samples were diluted 1:4, according to manufacturer's instructions, prior to analysis with luminex® performance assay for murine CCL2, GM-CSF, IFN-γ, IL-1β, IL-2, IL-4, IL-5, IL-6, IL-10, IL-12p70, IL-13, IL-17A, CXCL1, CXCL2, TNF-α and VEGF (R&D Systems, USA). Assays were, performed as per manufacturer's instructions and analysed on Bio-Rad® Bio-Plex analyser 200. For analysis, 5PL curves were used to fit standard curves with automated optimisation. Cytokine levels were compared using a Two-way ANOVA with a Bonfferoni post test comparing the PBS negative control and the SEB treated with CTLA4Ig therapy group to the SEB positive control group.

Summary of results: Pro-inflammatory cytokines/chemokines (FIG. 10, CXCL1; FIG. 11, IL-1β; FIG. 12, TNF-α) were significantly increased in the SEB positive control group as compared to the PBS negative control group. For CXCL1 and IL-1β3, these differences were only observed at day 3. CTLA4Ig therapy significantly reduced CXCL1 and IL-1β at 3 days post-exposure, suggesting that the therapy prevented the systemic response regarding these pro-inflammatory mediators. Interestingly, whilst TNF-α was raised in the SEB positive control group at 3, 6 and 14 days, CTLA4Ig administration did not significantly reduce TNF-α levels. This would suggest, contrary to current understanding, that TNF-α is not an important mediator to the clinical outcome of subjects exposed to SAgs as weight loss and clinical signs remain unaffected in the SEB treated with CTLA4Ig therapy group. Importantly, this would suggest that TNF-α is not a good biomarker for determining efficacy of B7 receptor antagonists for SEB-mediated disease, or potentially other disease states where this therapy is used.

In contrast, at day 3, levels of IFN-γ (FIG. 14) and IL-6 (FIG. 15) were significantly raised in the SEB positive control group but were reduced almost to baseline levels when CTLA4Ig was administered. This would suggest that IFN-γ and IL-6, and not TNF-α, are suitable biomarkers to monitor subject responsiveness to use of CTLA4Ig in the post-exposure treatment of SAg intoxication. This further indicates that IFN-γ and IL-6 are potentially appropriate biomarkers to evaluate subject responsiveness to CTLA4Ig, as well as other pharmaceutical composition active ingredients or agents capable of binding to B7 family receptors, in other disease states.

IL-2 (FIG. 13) was shown to be significantly raised in the SEB positive control group as compared to the PBS negative control group. While significant reduction to IL-2 was not seen for the SEB treated with CTLA4Ig therapy group, there was a trend in reduced IL-2 levels which suggests that CTLA4Ig may reduce T-cell proliferation.

At day 3, IL-10 (FIG. 16) was raised in the SEB positive control group as compared to the PBS negative control group. However, on days 6 and 14, the IL-10 response was equivalent to the PBS negative control group. Interestingly, there was a trend for increased IL-10 production by the SEB treated with CTLA4Ig therapy group at days 6 and 14 as compared to the SEB positive control group and PBS negative control group. This would suggest that CTLA4Ig allows the anti-inflammatory response to be induced after SEB exposure in order to reduce inflammation.

Splenocyte Isolation

Spleens were aseptically isolated from Balb/C mice, placed in 10 μm cell strainer and splenocytes pushed through the strainer into a sterile petri dish using the handle from a sterile cell scraper or sterile 10 ml syringe. The splenocyte cell suspension was added to a 50 ml falcon tube, brought up to a final volume of 30 ml with RPMI-1640 media containing 15% (v/v) fetal calf serum (Sigma-Aldrich, Poole, Dorset, UK), 1% (v/v) Penicillin/Streptomycin solution and 1% (v/v) L-Glutamine (Sigma-Aldrich, Poole, Dorset, UK) and centrifuged for 10 minutes at 1100 rpm. After centrifugation, the supernatant was discarded and the red blood cells lysed by adding 3 ml of red blood cell lysing buffer (Sigma, Dorset UK), mixing gently for 1 min. Sterile RPMI-1640 medium was added until the cell suspension final,volume was 30 ml, and the resulting solution was centrifuged for 10 min at 1100 rpm. After this centrifugation, the supernatant was discarded and 4 ml of sterile RPMI-1640 medium added. The pellet was re-suspended and the cells quantified using a Neubauer haemocytometer. The final suspension was then prepared to 1.0×10⁶ cells/ml in RPMI-1640 medium.

Proliferation MTT and SEB Cytotoxicity Assays

Isolated splenocytes were treated with PBS (negative control), SEB (positive control) or with SEB and CTLA4Ig and, after 24 hrs, cell proliferation and cell viability was measured using a modified MTT assay. This assay measured these parameters via the reduction of the active yellow tetrazolium MTT (3-(4, 5-dimethylthiazolyl-2)-2,5-diphenyltetrazolium bromide. Cells were plated at a seeding density of 1×10⁶ cells per well and incubated for 48 hr. 10 μl of the MTT reagent was added and the plate incubated for 4 hr. 100 μl detergent reagent was then added and the plates re-incubated for a further 3 hr. The plates were then read at 570 nms. For each of the 5 experiments the proliferation response of CTLA4Ig-treated cells were normalised against the negative control and the positive SEB control (0% and 100% respectively). Optical density was analysed using a Repeated Measure ANOVA with Dunnett's post test comparing the SEB treated with CTLA4Ig group to SEB positive control group and splenocyte only negative control group.

The ability of CTLA4Ig to inhibit the cytotoxic effect of SEB was investigated using a Promega MultiTox-Flour Multiplex Cytotoxicity Assay according to the manufacturer's instructions (Promega, USA). Cell toxicity assays were performed on 4 separate occasions: splenocytes only; splenocytes treated with CTLA4Ig; splenocytes exposed to SEB; and splenocytes exposed to SEB and treated with CTLA4Ig. Briefly, splenocytes were seeded into wells of 96-well flat bottomed cell culture plates at a density of 1×10⁶ cells per well (B. E. Thompson Supplies, Andover, UK). These cells were then exposed to 1.25×10⁻⁸M or 1.6×10⁻⁹M SEB in culture medium. Dead cell number were determined using fluorescence at 485Ex/535Em 3 hr after addition of the 5× MultiFlour reagent prepared according to the manufacturers guidance. Con A was also used as a positive proliferation control with a mean value of 366% increase in proliferation as compared to SEB (data not shown). Optical density was analysed using a Repeated Measure ANOVA with Dunnett's post test.

Summary of results: SEB characteristically causes a dose dependant proliferation of T-cells. Thus, primary cultures of isolated mouse splenocytes, which contain a high proportion of T-cells, were used as to measure SEB activity. CTLA4Ig significantly reduced splenocyte proliferation in response to SEB exposure across all concentration of CTLA4Ig (10-0.3125 μg/ml) (FIG. 17). The reduction in SEB-induced proliferation was in a dose-responsive manner and at the highest concentration reduced SEB proliferation to less than 40%. The reduction in proliferation was not due to therapeutic toxicity as CTLA4Ig-treated cells showed no cytotoxicity compared to splenocyte exposed to PBS only (FIG. 18).

ELISA for Cytokines IL-1β, IL-2 and MIP-1 Determination

Quantitative Quantikine ELISAs were performed to determine the effect of CTLA4Ig on CCL2, IL-1β, and IL-2 expression by murine splenocytes following SEB exposure. ELISA plates pre-coated for mouse CCL2, IL-1 β, IL-2 or MIP-1 were supplied by Quantikine. Standards, controls and samples were pipetted into the wells of the ELISA plate as per the manufacturer's instructions. Following removal of the supernatant from the plates used in the cytotoxicity study, the means of five experimental replicates was determined. CCL2, IL-1β and IL-2 levels expressed by splenocytes was analysed using a Repeated Measure ANOVA with Dunnett's post test comparing a range of CTLA4Ig treatment concentrations to SEB positive controls.

Summary of results: The SEB-induced expression of CCL2 (FIG. 19), IL-1β (FIG. 20) and IL-2 (FIG. 21) was significantly reduced when CTLA4Ig was administered to splenocytes at various concentrations. Importantly, the reduction of CCL2 and IL-1β suggests that CTLA4Ig intervention reduced SEB-induced inflammation. Additionally, the reduction observed for IL-2 suggests that CTLA4Ig reduces IL-2-induced T-cell proliferation when splenocytes are exposed to SEB.

1. A pharmaceutical composition for use in the post-exposure treatment of superantigen (SAg)-mediated disease in a subject, wherein the pharmaceutical composition comprises an active ingredient which is capable of binding an antigen presenting cell B7 receptor.

2. A pharmaceutical composition according to claim 1 administered as a single dose.

3. A pharmaceutical composition according to claim 1, wherein the active ingredient is selected from CTLA4Ig or derivatives thereof.

4. A pharmaceutical composition according to claim 3, wherein the active ingredient is CTLA4Ig.

5. A pharmaceutical composition according to claim 1, wherein the SAg is Staphylococcus Enterotoxin B.

6. A pharmaceutical composition according to claim 1, wherein the SAg-mediated disease is sepsis, toxic shock syndrome, infective endocarditis or necrotizing fasciitis.

7. A pharmaceutical composition according to claim 1, to be administered at least 3 hours post-exposure to SAg.

8. A pharmaceutical composition according to claim 1, to be administered at least 8 hours post-exposure to SAg.

9. A pharmaceutical composition according to claim 1, to be administered at least 144 hours post-exposure to SAg.

10. An agent capable of binding an antigen presenting cell B7 receptor for use in the post-exposure treatment of SAg-mediated disease.

11. An agent according to claim 10 administered as a single dose.

12. An agent according to claim 10, wherein the agent is CTLA4Ig, and derivatives thereof.

13. A method for monitoring subject responsiveness to post-exposure treatment of SAg-mediated disease with a pharmaceutical composition according to claim 1, the method comprising the steps of:
measuring the concentration of at least one of IFN-γ and IL-6 in a first and a second biological sample, wherein the first biological sample is from a subject pre-treatment, and the second biological sample is post-treatment;
comparing the pre- and post-treatment concentration of each respective biomarker;
and wherein a respective biomarker concentration lower in the post-treatment biological sample, relative to the pre-treatment biological sample, is a positive indicator of subject responsiveness to post-exposure treatment.
